(19) United States
(12) Patent Application Publication
(10) Pub. No.: US 2023/0404423 A1
(43) Pub. Date: Dec. 21, 2023

(54) APPARATUS AND METHOD FOR INTRAOPERATIVE REAL-TIME TUMOUR TISSUE DISCRIMINATION

(71) Applicant: IMPERIAL COLLEGE INNOVATIONS LIMITED, London (GB)

(72) Inventors: Timothy CONSTANDINOU, London (GB); Jinendra EKANAYAKE, London (GB); Yan LIU, London (GB); Steven WONG, London (GB)

(21) Appl. No.: 18/030,735

(22) PCT Filed: Oct. 13, 2020
(86) PCT No.: PCT/GB2020/052550

§ 371 Date: Apr. 6, 2023

(51) Int. Cl. A61B 5/0538; A61B 5/00

(52) U.S. Cl. CPC A61B 5/0538; A61B 5/7225; A61B 5/14551; A61B 2562/0247; A61B 2562/0219; A61B 5/7257

(57) ABSTRACT

Apparatus for discriminating between tumour tissue and non-tumorous tissue in real-time, the apparatus comprising: a handheld bioimpedance probe having an elongate body and at least four stimulator electrodes mounted on a distal end of the elongate body, the electrodes being arranged to be held against a region of tissue in use; a current source configured to generate a stimulation current; a voltage sensor; a multiplexer coupled between the current source, the voltage sensor and the stimulator electrodes and configured to changeably switch between a plurality of switching configurations, wherein, in each switching configuration, a first two of the electrodes are connected to the current source, and a second two of the electrodes are connected to the voltage sensor, the electrodes that constitute the first two electrodes and the electrodes that constitute the second two electrodes changing from one switching configuration to the next; and processor-controlled circuitry configured to: control the switching configuration of the multiplexer; control the generation of the stimulation current by the current source and the application of the stimulation current to the first two electrodes of each switching configuration, such that, in use, the stimulation current flows through the tissue between the first two electrodes; control the measurement, by the voltage sensor, of the voltage between the second two electrodes of each switching configuration, across the tissue in use; determine, based on the applied current and the measured voltage for each switching configuration, a measure of relative impedance of the tissue for each switching configuration; and supply such impedance measurements as output data to a data analyser to enable real-time discrimination of the tissue to be performed, based on the impedance measurements. A handheld surgical tool is also provided, comprising an elongate body and an adjustable handle member that is pivotally coupled to the elongate body.

### FIELD OF THE INVENTION

This invention relates to apparatus, and an associated method, for intraoperative real-time tumour tissue discrimination, to discriminate between tumour tissue and normal (non-tumorous) tissue. It is particularly applicable for the identification of brain tumours, although it is by no means limited to such an application, and may instead be used to discriminate between tumour tissue and normal tissue in other parts of the human (or potentially animal) body.

### BACKGROUND TO THE INVENTION

For effective diagnosis and treatment of cancers in the human (or potentially animal) body, the correct identification of tumours, and accurate discrimination between tumour tissue and normal (non-tumorous) tissue, is of great importance. Although the description that follows primarily relates to the determination of brain tumours during neurosurgery (i.e. neuro-oncology), the present disclosure is also applicable to the identification of tumour tissue in other parts of the human (or animal) body.

With particular regard to brain tumours, despite several advances in their management, tumours are either associated with high mortality and morbidity (as with gliomas), or present significant challenges due to disruption of normal physiology following surgery (as with pituitary tumours). Surgical resection is a frequently-used treatment for extrinsic and intrinsic brain tumours, with key aims being to include the most malignant tissue in the resected specimen, maximal resection, and avoiding or minimising removal of functional brain tissue [1].

Preservation of essential brain function combined with maximal, gentle and precise resection represents the best means of providing a surgical cure. In this regard, gross total resection (GTR) (>90% of macroscopically visible tumour) is directly associated with improved outcomes (progression free survival, overall survival) and quality of life. Further, supramaximal surgical resection (i.e. removing tissue beyond the intended resection margin to account for microscopic tumour invasion up to 2 cm beyond the ‘enhancing’ component of a tumour as visualised on contrasted MRI imaging) has additionally been associated with improved outcomes. However this must be balanced with preservation of essential cognitive and homeostatic function. Indeed, surgically-acquired functional impairments are directly linked to poorer prognoses. Therefore, the corresponding challenge to maximal tumour resection in neuro-oncology surgery is the preservation of normal functioning tissue. This can be difficult, given that tumours may not be clearly distinguishable on the basis of visual inspection alone during surgery; tumours invade normal tissue at a microscopic level with even a small volume of cells being relevant, involving normal tissue in a ‘mosaic’ fashion. A further consideration is that brain tissue which has been invaded by tumour may still have function. Therefore, identifying and establishing the boundary of normal and abnormal brain tissue is a fundamental goal of resective brain tumour surgery.

Tumour identification can in part be achieved through the use of pre-operative neuroimaging, e.g. high-resolution MRI, which is integrated with on-table ‘neuronavigation’— a type of intraoperative anatomical ‘GPS’. However, a limitation of these approaches are that they are ‘macroscopic’. Further, they are usually acquired pre-operatively or preoperatively, and as such are susceptible to real-time distortions of the brain anatomy during surgical resection, as a result of tumour removal and brain ‘shift’.

There is, therefore, currently an unmet need in the field of neuro-oncology for a simple, practical and robust means of on-table ‘real-time’ differentiation of normal brain tissue from abnormal brain tissue. More particularly, such a real-time intraoperative technique should enable the detection of the surgical margin (i.e. distinguishing between healthy brain tissue and a tumour) during both resective and biopsy procedures. Such a technique has the potential to improve the quality of neurosurgical treatment, optimising tumour identification and reducing excessive damage to healthy brain tissue [2]. This would ultimately reduce the mortality rate associated with brain cancer recurrence, along with financial and quality-of-life burdens to the patient and healthcare provision linked to cancer recurrence. Real-time differentiation of brain tumour tissue from normal tissue facilitates (a) preservation of brain function by avoiding removal of normal or predominantly normal brain tissue; and (b) increased precision and accuracy in removing abnormal brain tissue, thereby facilitating GTR of the brain tumour.

Techniques which provide real-time visualisation during surgical resection are the focus of significant investigation. Current approaches include hyperspectral imaging, confocal microscopy, intraoperative ultrasound-guided surgery (USS), and intraoperative Raman spectroscopy. The latter two technologies are currently in formal clinical use. Intraoperative USS is widely used but remains user dependent and vulnerable to difficulties in interpretation. This is particularly the case when required to identify anything other than gross macroscopic tissue abnormalities. Intraoperative Raman spectroscopy has recently been translated into the neurosurgical operating theatre in the context of a clinical trial with the promise of providing high resolution, microscopic identification of tumour cells [3].

Bioimpedance is one of the most promising means of evaluating the physiological state of different tissue types, resulting from the biological structure of tissues causing impedance differences. Bioimpedance techniques involve passing an electrical current through biological tissue while concurrently measuring the resistance to the flow of current produced by the tissue. It has been previously reported [4] that there are significant differences in bioimpedance of healthy and cancerous tissue in liver, prostate, breast, tongue and etc. in both in-vivo and ex-vivo measurements [5]-[6]. However, existing bioimpedance apparatus is primarily in the form of laboratory-based bench-top equipment, and is therefore not well suited to characterising bioimpedance in real-time, intraoperatively, for the purpose of enabling a surgeon to differentiate normal brain tissue from abnormal brain tissue (i.e. brain tumour tissue) during the course of surgery.

There is therefore a need for straightforward and effective bioimpedance characterisation apparatus that can conveniently be used by a surgeon to provide real-time intraoperative bioimpedance-based information, to enable the surgeon to differentiate normal brain tissue from abnormal brain tissue during the course of surgery, and thereby address at least some of the above problems. Other problems addressed by the present disclosure will become apparent from the description below and the accompanying drawings.

A related challenge we address in the field of hand-held surgical tools is the optimisation of the surgical handle to enhance frequently used hand-grips. There are three cardinal hand positions when holding a linear pen-like instrument for artistic or surgical purposes. They include (1) a ‘pencil’ grip, with the first three fingers of the hand forming a ‘tripod’ around the tool; (2) a ‘painter's’ grip, where one end of the tool is held lightly primarily by the first two fingers; and (3) an ‘overhand extended’ grip stabilised by an extended index finger. Each of these grips offers specific improvements in precision, freedom of movement and stability relative to the other grips. Despite the long-standing use of these different grips in a variety of fields, there is an absence of a single dedicated tool handle which allows changes in position to facilitate and fortify a specific grip.

### SUMMARY OF THE INVENTION

According to a first aspect of the present invention there is provided apparatus for discriminating between tumour tissue and non-tumorous tissue in real-time, as defined in claim 1 of the appended claims.

With the present invention, we target an existing intraoperative clinical tool for the purpose of real-time tumour tissue identification taking advantage of its established use in the intraoperative neurosurgical work-flow for tumour resection. Intraoperative direct cortical stimulation is used in awake and asleep brain surgery to identify functionally active brain tissue. The present invention expands on this principle, providing a handheld probe device to enable the application of bioimpedance for the purposes of real-time differentiation of brain tumour tissue from normal brain tissue.

More particularly, there is provided apparatus for discriminating between tumour tissue and non-tumorous tissue in real-time, the apparatus comprising:
a handheld bioimpedance probe having an elongate body and at least four stimulator electrodes mounted on a distal end of the elongate body, the electrodes being arranged to be held against a region of tissue in use;
a current source configured to generate a stimulation current;
a voltage sensor;
a multiplexer coupled between the current source, the voltage sensor and the stimulator electrodes and configured to changeably switch between a plurality of switching configurations, wherein, in each switching configuration, a first two of the electrodes are connected to the current source, and a second two of the electrodes are connected to the voltage sensor, the electrodes that constitute the first two electrodes and the electrodes that constitute the second two electrodes changing from one switching configuration to the next; and
processor-controlled circuitry configured to:
   control the switching configuration of the multiplexer;
   control the generation of the stimulation current by the current source and the application of the stimulation current to the first two electrodes of each switching configuration, such that, in use, the stimulation current flows through the tissue between the first two electrodes;
   control the measurement, by the voltage sensor, of the voltage between the second two electrodes of each switching configuration, across the tissue in use;
   determine, based on the applied current and the measured voltage for each switching configuration, a measure of relative impedance of the tissue for each switching configuration; and
   supply such impedance measurements as output data to a data analyser to enable real-time discrimination of the tissue to be performed, based on the impedance measurements.

The term “stimulation current” should be interpreted broadly, to encompass AC waveforms or potentially a DC signal; it may in practice be any arbitrary current.

By virtue of the bioimpedance probe being handheld and having an elongate body, this enables the probe to be conveniently used by a surgeon (or conceivably a robotic arm) to provide intraoperative bioimpedance-based information, preferably in a manner that does not obstruct the surgeon's view of the tissue region in question. Moreover, by virtue of having at least four stimulator electrodes and the aforementioned configuration and manner of operation of the multiplexer, impedance measurements can be obtained quickly and with a high degree of precision.

In certain embodiments the current source is a voltage controlled current source, and the processor-controlled circuitry further comprises a voltage waveform generator configured to generate a voltage waveform and to supply the voltage waveform to the current source. The current source may further comprise a high pass filter configured to remove DC offset from the voltage waveform and to convert the voltage waveform to the stimulation current. Such a high pass filter may not be required if the input of the voltage controlled current source is differential or bipolar.

Alternatively, a current waveform generator may be used, to generate a current waveform to directly drive the current source.

The voltage waveform generator or current waveform generator may comprise a digital-to-analogue converter configured to receive a predefined stimulation waveform from which the voltage waveform or current waveform is generated.

Alternatively, the voltage waveform generator or current waveform generator may comprise a Direct Digital Synthesis module configured to receive a predefined stimulation waveform from which the voltage waveform or current waveform is generated.

The stimulation waveform may be stored in the processor-controlled circuitry.

Preferably the voltage waveform or current waveform comprises a mix of a plurality of different frequencies, thereby enabling the tissue impedance at the different frequencies to be rapidly extracted using Fast Fourier Transform processing (or another appropriate technique). This greatly reduces the impedance measurement time compared to a standard chirp technique that would employ frequency sweeping, and also improves measurement accuracy. Preferably the voltage waveform or current waveform has substantially equal magnitude at each of the plurality of different frequencies.

Preferably the voltage sensor comprises an amplifier, such as an instrumentation amplifier, or a differential amplifier.

Preferably the processor-controlled circuitry further comprises an analogue-to-digital converter configured to receive and sample a voltage signal from the amplifier and thereby generate digital voltage data.

For embodiments in which the voltage waveform comprises a mix of a plurality of different frequencies, the processor-controlled circuitry may further comprise a Fast Fourier Transform processor configured to receive the digital voltage data from the analogue-to-digital converter, and to convert time-domain data into frequency-domain data and thereby extract the instantaneous magnitude of the voltage data for each frequency in the stimulation waveform.

The multiplexer may be configured to cyclically switch between each of the plurality of switching configurations.

The processor-controlled circuitry may comprise a microcontroller.

In certain embodiments the voltage controlled current source and/or the amplifier are provided on a front-end printed circuit board or integrated circuit within the probe.

The digital-to-analogue converter and/or the analogue-to-digital converter may be provided on a microcontroller unit (MCU) platform.

The data analyser may comprise a personal computer (PC), or another suitable device such as a tablet computer or smartphone.

In certain embodiments the electrodes may be spherical or hemispherical, and/or may be spring-loaded. Alternatively the electrodes may for example be needle-shaped or pointed.

In certain embodiments the distal end of the elongate body may comprise a telescopic shaft on which the electrodes are mounted. Such an arrangement enables or facilitates endonasal access of the probe to the brain.

The probe may further comprise an adjustable handle member at a proximal end of the elongate body. More particularly, the handle member may be pivotally coupled to the elongate body. This advantageously enables the probe to be held more stably in the surgeon's hand, e.g. in either an ‘overhand’ grip stabilised by an extended index finger (with the handle held in the palm at an ‘angled’ setting) or in a ‘pencil’ grip.

In certain embodiments the handle member is pivotally and retractably coupled to the elongate body by means of a linkage mechanism comprising a first joint mounted on or in the handle member, a second joint mounted on or in the elongate body, and a connecting rod that extends from the first joint to the second joint and passes through at least one of the first and second joints to an adjustable extent, wherein at least one of the first and second joints comprises a ball-and-socket joint to enable rotation in three dimensions. For example, the second joint may comprise a ball-and-socket joint and the rod may extend through the second joint to an adjustable extent. Alternatively, or in addition, the first joint may comprise a ball-and-socket joint and the rod may extend through the first joint to an adjustable extent.

Preferably, in the or each ball-and-socket joint, the surface of the respective ball part and/or the surface of the respective socket is adapted to hold the rotational position of the ball part relative to the socket in a position as set by the user.

In certain embodiments the probe may further comprise an operation button mounted on the elongate body, operable to activate the processor-controlled circuitry.

The probe may further comprise depressions on the elongate body, for receiving the user's fingertips in use, in order to further stabilise the instrument.

In certain embodiments the probe may be wireless, to facilitate unencumbered manipulation of the probe by the surgeon.

In certain embodiments the probe may further comprise a pressure sensor, for measuring the contact pressure between the electrodes and the tissue in use.

In certain embodiments the probe may further comprise a blood oxygen sensor at the distal end of the elongate body, for measuring the blood oxygen level of the tissue in use.

In certain embodiments the probe may further comprise an accelerometer or inertial sensor to sense the angle of inclination of the probe.

According to a second aspect of the present invention there is provided a handheld surgical tool comprising an elongate body and an adjustable handle member that is pivotally coupled to the elongate body. Application of the adjustable handle member is by no means limited to the present bioimpedance probe; the principles of the adjustable handle member are also applicable to other surgical tools having an elongate body for which a more stable hold is desired. Optional features of the adjustable handle member are as outlined above in relation to the first aspect of the invention.

According to a third aspect of the present invention there is provided a method of discriminating between tumour tissue and non-tumorous tissue in real-time, using the apparatus according to the first aspect of the invention.

### BRIEF DESCRIPTION OF THE DRAWINGS

Embodiments of the invention will now be described, by way of example only, and with reference to the drawings in which:

FIGS. 1a and 1 b illustrate side views of a handheld bioimpedance measurement probe having an adjustable (pivotable and retractable) handle member, an elongate shaft-like body, and a stimulator end (which may optionally be telescopic) having a plurality of stimulation electrodes;

FIG. 2a illustrates another side view of the bioimpedance measurement probe;

FIG. 2b illustrates an enlargement of region A of FIG. 2a, showing a close-up of the stimulator end and four stimulation electrodes;

FIGS. 3a, 3b and 3c illustrate, respectively, a top view, side view and end-on view of the bioimpedance measurement probe;

FIG. 4a illustrates another side view of the bioimpedance probe, which includes an operation button and finger-grip depressions;

FIG. 4b illustrates an enlargement of region A of FIG. 4a, showing a close-up of the operation button and finger-grip depressions;

FIGS. 5a and 5b illustrate cross-sections of a linkage mechanism for pivotally and retractably coupling the handle member to the elongate body of the bioimpedance measurement probe, in retracted and extended configurations respectively;

FIGS. 6a to 6d illustrate further cross-sections of the linkage mechanism of FIGS. 5a and 5b, in a variety of configurations;

FIG. 7 illustrates a variant of the adjustable handle and pivotal linkage mechanism of FIGS. 5a and 5b;

FIG. 8 illustrates possible angular positions into which the handle of FIG. 7 may be adjustably set, by means of the pivotal linkage mechanism;

FIGS. 9a, 9b and 9c illustrate further possible positions into which the handle of FIG. 7 may be adjustably set, by means of the pivotal linkage mechanism;

FIG. 10 illustrates a block diagram of an example bioimpedance characterisation system for use with, or incorporating, the above bioimpedance measurement probe;

FIG. 11 illustrates example front-end analogue circuit schematics for the system of FIG. 10, including a voltage controlled current source, a voltage buffer, an instrumentation amplifier, and a multiplexer to which the stimulation electrodes of the bioimpedance measurement probe are connected;

FIG. 12 illustrates an electrode switching sequence in respect of a bioimpedance measurement probe having four stimulation electrodes, in which four different electrode configurations are cycled through;

FIG. 13 illustrates a variant of the electrode switching sequence of FIG. 12, again in respect of a bioimpedance measurement probe having four stimulation electrodes, but in which six different electrode configurations are cycled through;

FIG. 14 illustrates examples of how the aforementioned probe configuration employing four-electrode measurement can be extended to more electrodes;

FIG. 15 schematically illustrates a multiplexer switching arrangement for supplying a current to a selected pair of stimulation electrodes when cycling though a series of electrode configurations;

FIG. 16 illustrates a voltage waveform and a corresponding FFT frequency response output, showing that the voltage waveform comprises multiple frequencies;

FIG. 17 is a flow diagram in respect of the operation of the bioimpedance characterisation system and measurement probe;

FIG. 18a illustrates a linearity characterisation of an example implementation of the bioimpedance characterisation system, from 0.2Ω to 500Ω;

FIG. 18b illustrates noise analysis of the bioimpedance characterisation system on a 25Ω discrete resistor with a sampling frequency of 192 kSample/s;

FIG. 19 illustrates reconstructed resistance mapping in respect of a region of metal surrounded with saline solution, performed using (a) an Agilent E4980A Precision LCR meter, and (b) a bioimpedance characterisation system and measurement probe according to the example implementation of the invention (as per the described Example 1);

FIG. 20 illustrates four measured voltage waveforms (as per the described Example 2);

FIG. 21 illustrates the FFT frequency response corresponding to each of the waveforms of FIG. 20;

FIG. 22 illustrates the detection of a biological tissue region with higher bioimpedance relative to a tissue region of lower bioimpedance;

FIG. 23 illustrates four measured voltage waveforms (as per the described Example 3);

FIG. 24 illustrates the FFT frequency response corresponding to each of the waveforms of FIG. 23;

FIG. 25 illustrates the detection of a biological tissue region with higher bioimpedance relative to a tissue region of lower bioimpedance; and

FIG. 26 illustrates (a) a piece of rib-eye steak (acting as a biological sample), and (b) reconstructed impedance mapping of the different tissue types (fat vs muscle fibre) across the piece of rib-eye steak, performed using the abovementioned example implementation of the invention (as per the described Example 4).

In the figures, like elements are indicated by like reference numerals throughout.

### DETAILED DESCRIPTION OF PREFERRED EMBODIMENTS

The present embodiments represent the best ways known to the Applicant of putting the invention into practice. However, they are not the only ways in which this can be achieved.

The present disclosure provides apparatus for discriminating between tumour tissue and non-tumorous tissue in real-time using bioimpedance measurements.

More particularly, through measuring the electrical properties of biological tissue of the brain tumour, we are able to identify a difference between normal and abnormal brain tissues. Further, impedance measurements are expected to enable differentiation between less aggressive and more aggressive tumour tissue. By evaluating these bioimpedance properties intraoperatively, clinically relevant information regarding surgical margin can be established in real-time [7].

Bioimpedance Probe—Physical Architecture

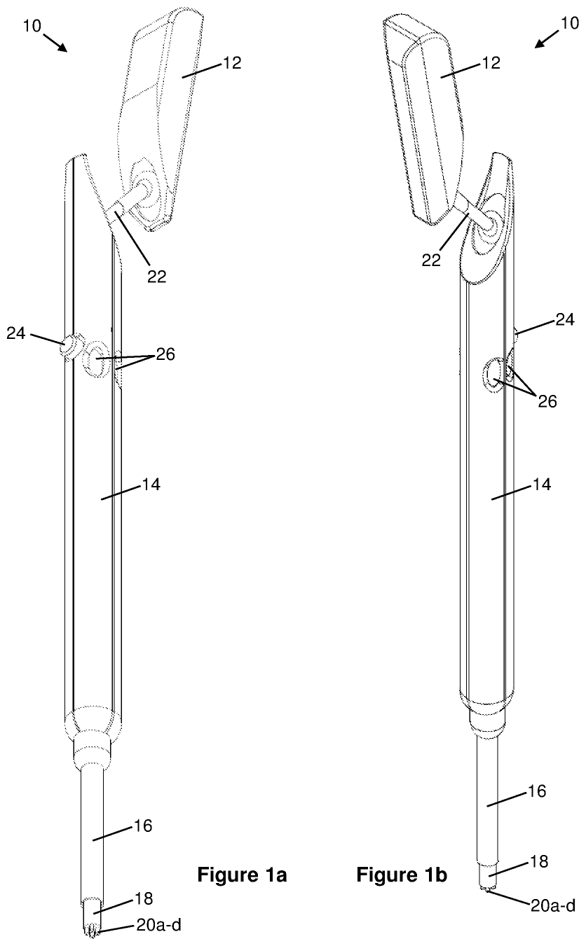

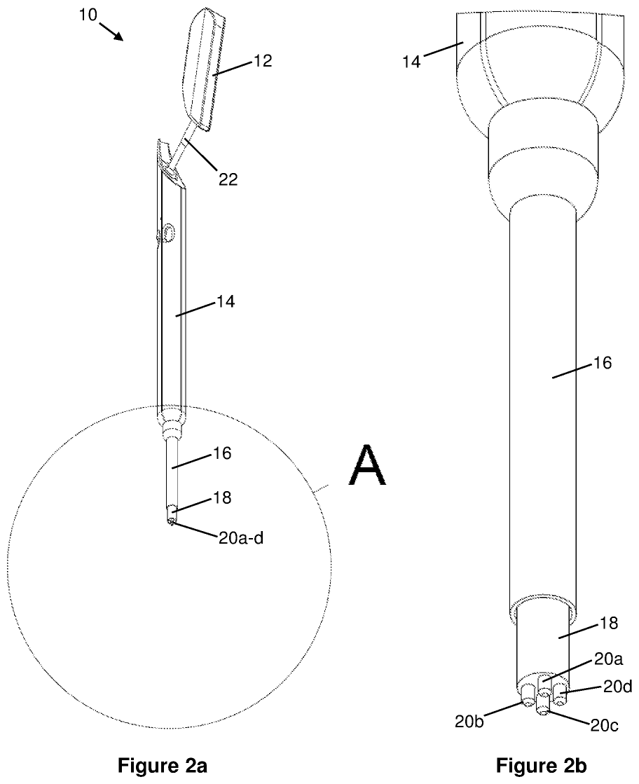

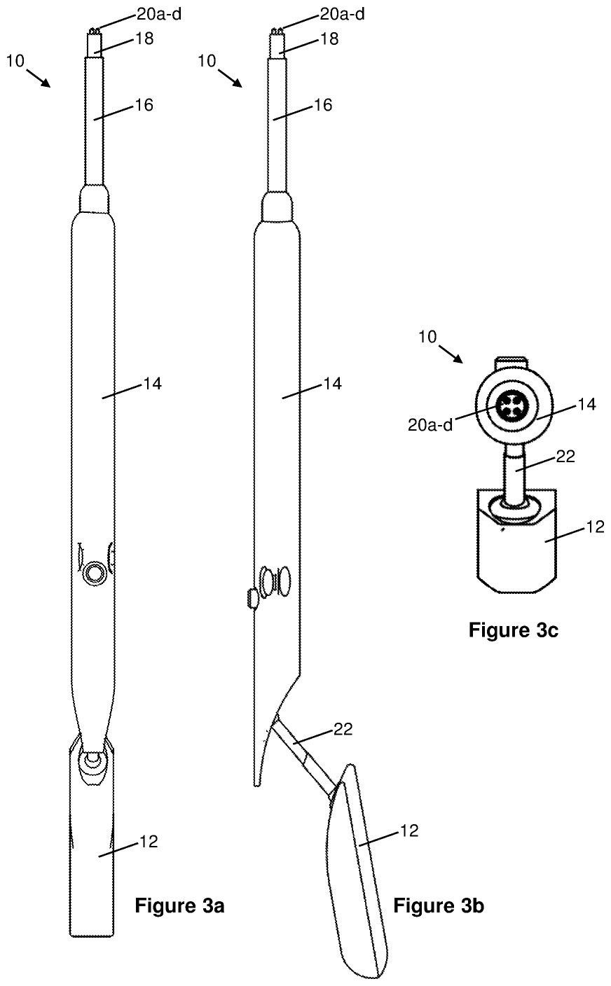

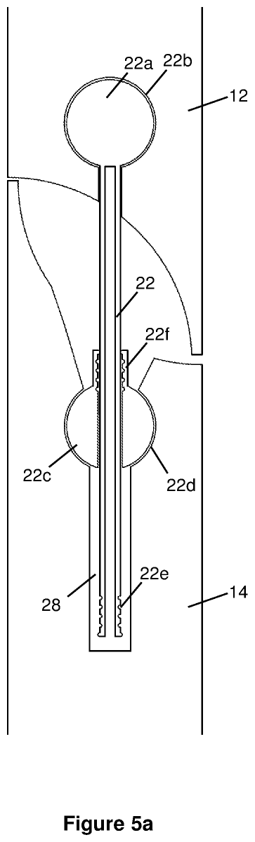

With reference initially to FIGS. 1a, 1 b, 2a, 2b, 3a, 3b, 3c, 4a and 4b, the present apparatus comprises a handheld bioimpedance probe 10 having an elongate body 14, at least four stimulator electrodes 20a-20d mounted on a distal end of the elongate body 14, and, optionally, an adjustable handle member 12 (that will be described in greater detail below with reference to FIGS. 5a to 9c). In various embodiments, the electrodes 20a-20d may be relatively long (e.g. pin-like, needle-shaped or pointed) or relatively short (e.g. stubby), and may be spherical or hemispherical in shape. In use, the tips of the electrodes 20a-20d are held against a region of tissue that is to be investigated for the presence of tumours, for the purpose of discriminating between tumour tissue and normal (non-tumorous) tissue. Optionally but advantageously, the electrodes 20a-20d may be spring-loaded so as to cushion their contact with the tissue being tested, and reduce the extent to which they press into the tissue.

In the illustrated embodiment the stimulator electrodes 20a-20d are mounted at the end of an optional telescopic shaft 18, that is extendable from, and retractable into, a cylindrical sleeve 16 at the distal end of the body 14. The length of the telescopic shaft 18 that protrudes from the cylindrical sleeve 16 can be lengthened or shortened and then fixed, to produce a desired case-specific or surgeon-specific length, thereby facilitating user-optimised application. An adjustment mechanism for adjusting and locking (or unlocking) the protruding length of the telescopic shaft 18 may be provided, as those skilled in the art will appreciate. The telescopic end of the probe 10 enables or facilitates endonasal access to the brain; for this the entire probe may be of a more elongated structure.

The optional adjustable handle 12 may incorporate, or be made from, a padded or malleable material, and can be placed in the surgeon's palm, creating an ‘angled grip’ of the probe 10. Alternatively, the adjustable handle 12 can be positioned above the first web space of the hand, thereby creating a ‘pencil grip’ of the probe 10. In either case, this helps the surgeon achieve greater control and precision grip of the probe 10.

More particularly, the adjustable handle 12 may be bent down, at an angle from the elongate body 14, to fit into the user's palm, with the user's fingers forming an encircling grip around the back of the instrument. The angulation of the handle 12 with respect to the body 14 produces specific utility as an ‘angled’ grip. Alternatively the handle 12 can be brought up and locked into place, in line with the body 14, enabling a ‘pencil’ grip.

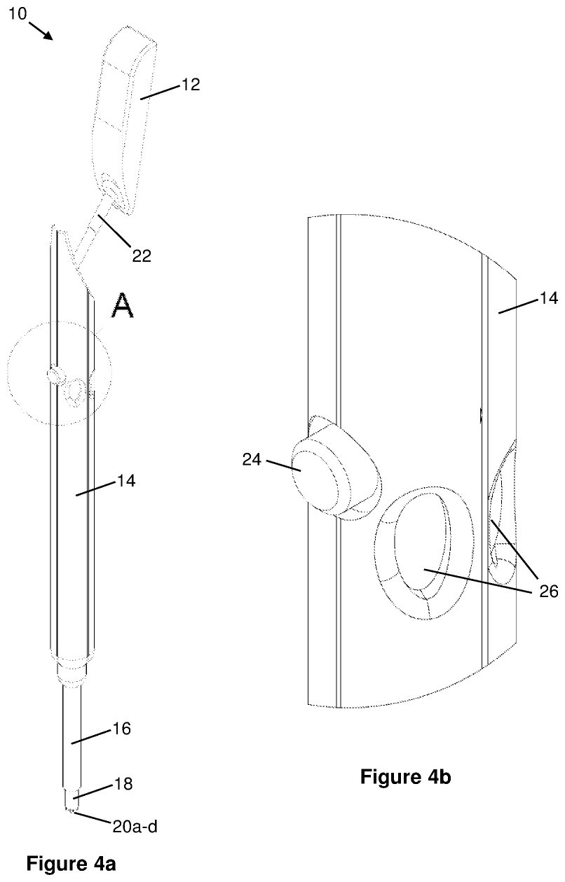

An operation button 24 (shown in close-up in FIG. 4b) is mounted on the elongate body 14, near to the finger depressions 26, for operation by the surgeon's index finger. Pressing the button 24 causes the probe 10 to operate, and releasing the button 24 causes operation of the probe to stop.

Bilateral finger depressions 26 are provided on the elongate body 14, either side of the operation button 24, to enable further stabilisation of the instrument, particularly when in the ‘angled grip’ position (which is expected to have specific utility for endonasal use).

The overall design of the elongate handheld probe 10 is such that it is low-profile and light in weight, rendering it suitable for use in delicate microsurgical procedures, or minimally-invasive brain surgery (e.g. microscope-based, endonasal or endoscopic surgery), to provide the surgeon with a real-time ‘look-ahead’ differentiation between abnormal and normal brain tissue.

Adjustable Handle

As mentioned above, the probe 10 may be provided with an adjustable handle member 12, to enable the probe 10 to be held more stably in the surgeon's hand. In the presently-preferred embodiments the handle member 12 is pivotally and retractably coupled to the elongate body 14 of the probe by means of a linkage mechanism.

FIGS. 5a and 5b illustrate cross-sections of the linkage mechanism for pivotally and retractably coupling the handle member 12 to the elongate body 14 of the probe 10, in retracted and extended configurations respectively.

The linkage mechanism comprises a first joint (22a, 22b) mounted on or in the handle member 12, a second joint (22c, 22d) mounted on or in the elongate body 14, and a connecting rod 22 that extends from the first joint to the second joint. In the illustrated embodiment the first joint is a ball-and-socket joint comprising a ball 22a on a first end of the rod 22, and a socket 22b in the handle 12 into which the ball 22a locates. Similarly, the second joint is a ball-and-socket joint comprising a ball 22c on the second end of the rod 22, and a socket 22d in the body 14 into which the ball 22c locates. At least one of the first and second joints (in the illustrated case, the second joint) is rotatable.

In each ball-and-socket joint, the surface properties between the ball part and its respective socket are preferably such as to enable the rotational position of the ball relative to the socket to be ‘fixed’ by the user as desired. For example, dimples/pimples or some other detent mechanism may be incorporated on the outer surface of the ball and/or the inner surface of the socket. Alternatively the ball-and-socket joint may employ a snug/tight friction-based fit, or some other kind of sticky interface between the ball and the socket.

To permit extension and retraction of the handle 12 relative to the body 14, the rod 22 passes through at least one of the first and second joints (in the illustrated case, the second joint and not the first) to an adjustable extent. Accordingly, a channel 28 is provided within the body 14 to accommodate the rod 22 when in the retracted state. The first end of the rod 22 is fixedly attached to the ball 22a of the first joint, whereas the second end of the rod 22 is non-fixedly (e.g. slidingly) coupled to the ball 22c of the second joint.

The second end of the rod 22 is provided with a gripping region 22e that, at the point of maximum extension, engages with a gripping/restraining collar 22f mounted on the ball 22c and prevents the rod 22 from separating from the ball 22c.

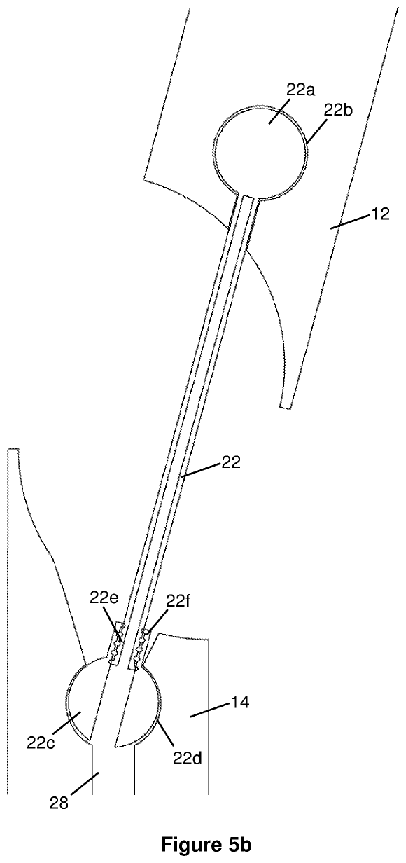

FIGS. 6a to 6d illustrate further cross-sections of the linkage mechanism of FIGS. 5a and 5b, in a variety of configurations. More particularly, FIG. 6a shows the handle 12 fully retracted, close to the body 14, with the rod 22 accommodated in the channel 28 within the body 14. FIG. 6b shows the handle 12 fully extended, away from the body 14, in a linear manner. FIG. 6c shows the extended handle 12 angled to one side by rotation of the ball 22c within the socket 22d in one direction, and FIG. 6d shows the extended handle 12 angled to the other side by rotation of the ball 22c within the socket 22d in the opposite direction.

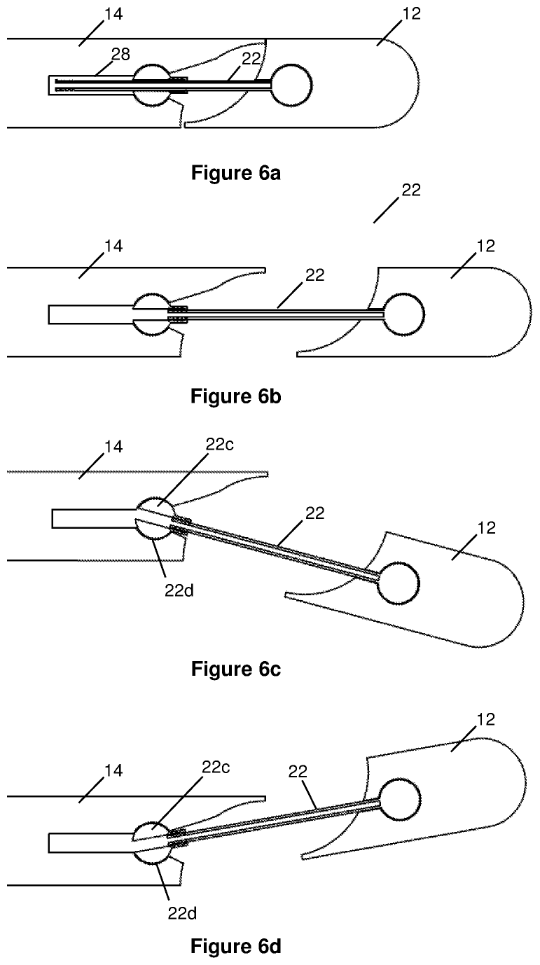

FIG. 7 illustrates a variant of the adjustable handle and pivotal linkage mechanism of the bioimpedance measurement probe. In this variant, in the two ball-and-socket joints, both the balls 22a and 22c are rotatable relative to their respective sockets 22b and 22d (the latter not being visible in the figure). The connecting rod 22 may again be telescopic.

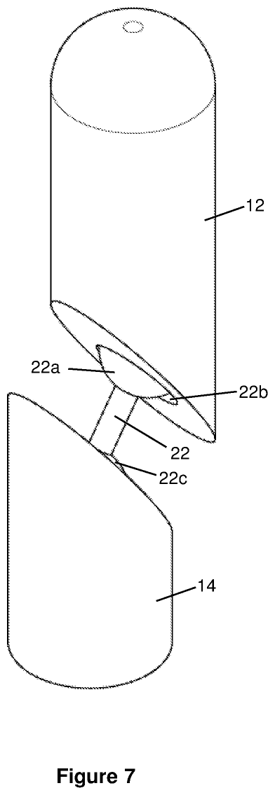

FIG. 8 illustrates possible angular positions into which the handle 12 of FIG. 7 may be adjustably set, by means of the pivotal linkage mechanism, by rotation of the ball 22a within the socket 22b. The double-headed curved arrow represents angular adjustment of the handle 12 between the two angular positions illustrated.

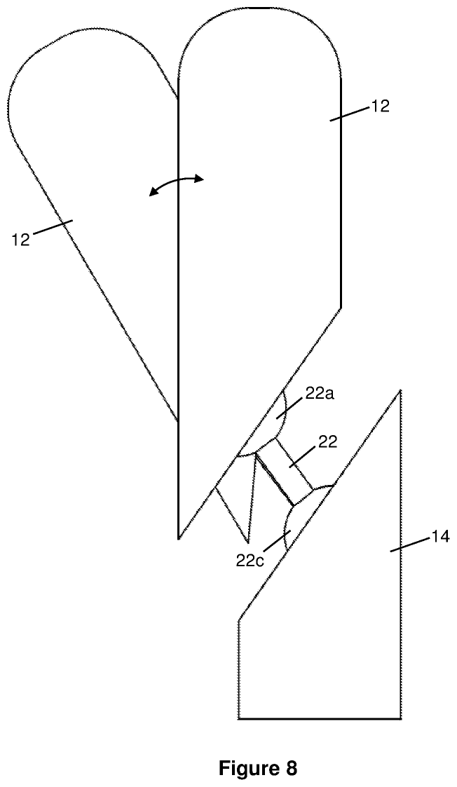

FIGS. 9a, 9b and 9c illustrate possible extended and retracted positions into which the handle of FIG. 7 may be adjustably set, by means of the pivotal linkage mechanism. More particularly, FIG. 9b shows the handle 12 in an extended position relative to the body 14, whereas FIG. 9c shows the handle 12 in a retracted position relative to the body 14. FIG. 9a shows both the extended and retracted positions overlaid on one another.

It will be appreciated that application of the adjustable handle member 12 is by no means limited to the present bioimpedance probe 10, and that the principles of the adjustable handle member 12 of FIGS. 5a to 9c are also applicable to other surgical tools having an elongate body, for which a more stable hold is desired, such as a needle driver or scalpel blade.

Bioimpedance Characterisation System Electronics

We have identified the essential requirements for an intraoperative impedance probing system. To prove the concept, an example PCB-based system has been designed and implemented with an on-board current source generator and voltage amplification circuits. A tetrapolar impedance probe based on four electrodes 20a-20d has been used and impedance measurements across electrode pairs have been characterised via I-V (current-voltage) measurement using a multitone sinusoidal current waveform. Measured results have then been processed by a microcontroller and host PC platform to identify the impedance values and reconstruct an impedance map in respect of the test samples. These results have been compared with a commercially-available Agilent E4980A Precision LCR instrument to benchmark performance.

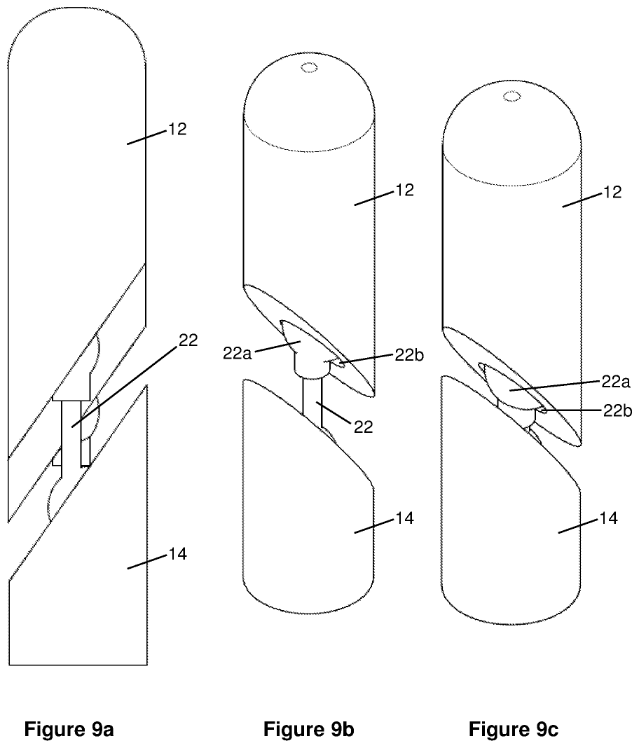

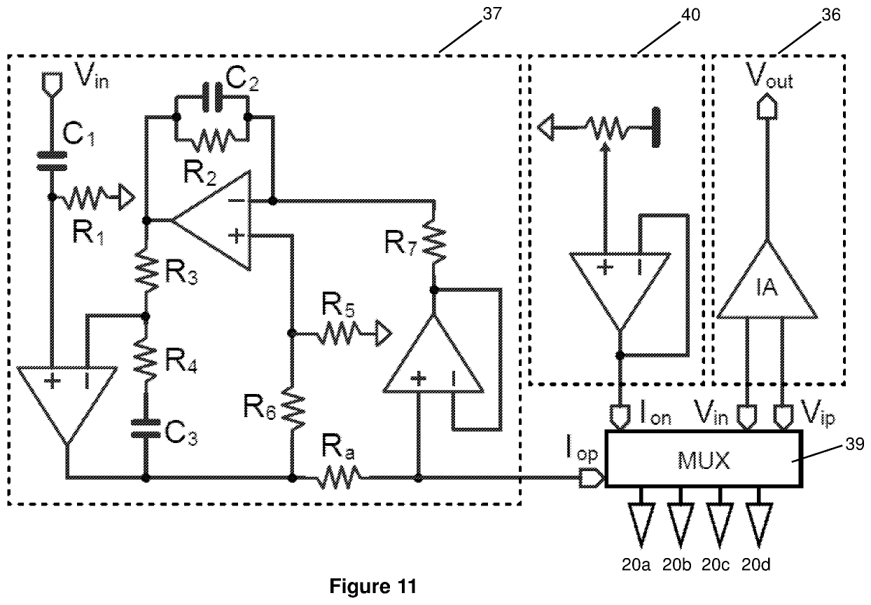

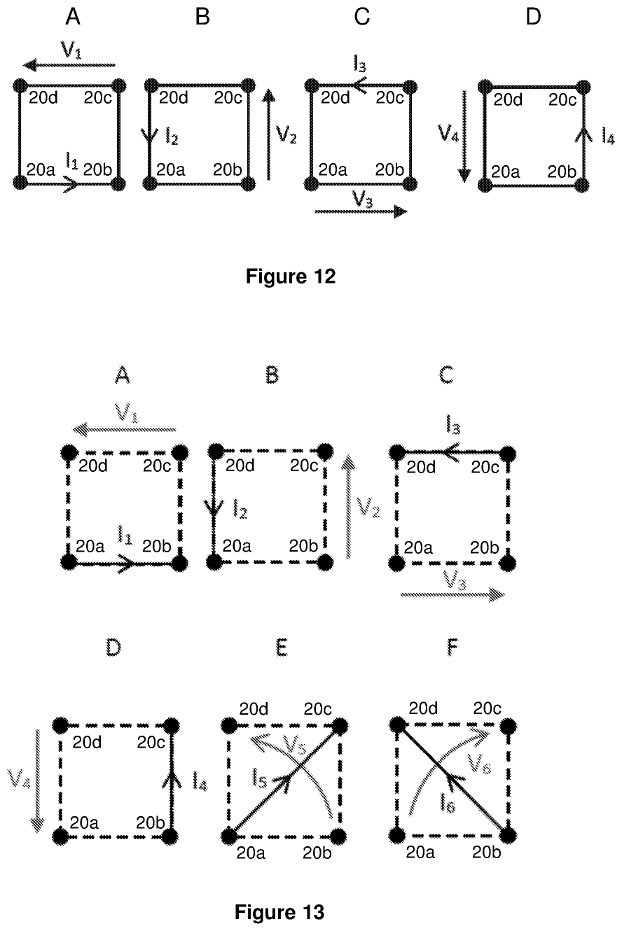

With reference initially to FIG. 10, in a general sense the present apparatus comprises (in addition to a probe 10 as described above, having at least four stimulator electrodes 20a-20d at its distal end): a current source 37 configured to generate a stimulation current; a voltage sensor (e.g. instrumentation amplifier) 36; and a multiplexer 39 coupled between the current source 37, the voltage sensor 36 and the stimulator electrodes 20a-20d and configured to changeably (preferably cyclically) switch between a plurality of switching configurations, such as those described below with reference to FIGS. 12 and 13.

In each switching configuration, a first two of the electrodes are connected to the current source 37, and a second two of the electrodes are connected to the voltage sensor 36, the electrodes that constitute the first two electrodes and the electrodes that constitute the second two electrodes rapidly changing from one switching configuration to the next.

Processor-controlled circuitry is also provided, configured to:
control the switching configuration of the multiplexer 39 (by application of a control signal to the multiplexer);
control the generation of the stimulation current by the current source 37 and the application of the stimulation current to the first two electrodes of each switching configuration, such that, in use, the stimulation current flows through the tissue between the first two electrodes;
control the measurement, by the voltage sensor 36, of the voltage between the second two electrodes of each switching configuration, across the tissue in use;
determine, based on the applied current and the measured voltage for each switching configuration, a measure of relative impedance of the tissue for each switching configuration; and
supply such impedance measurements as output data to a data analyser to enable real-time discrimination of the tissue to be performed, based on the impedance measurements.

The impedance of tissue cell is well studied for cellular processes or brain tissue [8]. However, the distribution of impedance of brain tumour is not conclusive. A recent study stated the average impedance meningiomas, low-grade gliomas, and high-grade gliomas are 530 Ω-cm, 160 Ω-cm, and 498 Ω-cm respectively [8]. Therefore, we have determined that the target impedance range should ideally cover 100 Ω-cm to 600 Ω-cm.

The present four-electrode measurement method is used due to it not being affected by polarisation (unlike a two-electrode method, which would be affected by polarisation). For safety reasons, the stimulation current driving the electrodes 20a-20d should of course be in compliance with appropriate medical device regulations, and is typically less than 10 μA. Considering the impedance of normal and cancerous brain tissues, the voltage level received at the sensing electrodes is typically in the range of a few millivolts, depending on the stimulation current used and the spacing of the electrodes.

To enable real-time intraoperative impedance characterisation, the processing time should be minimised. Therefore the control electronics (e.g. microcontroller unit (MCU) 31) should be capable of converting the analogue signal if an on-board analogue-to-digital converter 32 is used, and extracting frequency domain signals by using Fast Fourier Transform (FFT) or envelope detection. As saline solution has to be poured over the cortex of the brain frequently to avoid drying, an analogue multiplexer 39 is preferably used to switch between different measuring configurations to ensure rapid measurement time and consistent measurements recorded over each configuration.

System Architecture

In more detail, a block diagram of the electronics system of our example implementation is shown in FIG. 10. It consists of four main parts:

1) A compact handheld probe (probe 10 described above) with multi-pole electrodes 20a-20d to perform versatile impedance characterisation configuration.

2) A front-end printed circuit board 35 (or integrated circuit), which may be incorporated within the probe 10, or provided in a separate interface unit to which the probe 10 (specifically the electrodes 20a-20d thereof) is connected. In this example the front-end board 35 includes a voltage controlled current source 37 to generate the stimulation current; an instrumentation amplifier 36 to sense the voltage signal; and a multiplexer array 39 for switching the current source 37 and the instrumentation amplifier 36 between electrodes. As illustrated, a Direct Digital Synthesis (DDS) module 38 may also be provided. A standalone ADC or DAC may be added to alleviate the processing burden in the microcontroller unit (MCU) 31. However, as mentioned below, in other embodiments the current source may directly generate a stimulation current for driving the electrodes 20a-20d, with the current source being driven by a current waveform generator (e.g. a current DAC).

3) An MCU platform 31 to control the standalone waveform generator or ADC, and pre-process the data. This may also be incorporated within the probe 10, or provided in a separate interface unit to which the probe 10 (specifically the electrodes 20a-20d thereof) is connected. In this example the MCU platform 31 includes a digital-to-analogue converter (DAC) 33 which is configured to generate a voltage waveform and to supply the voltage waveform to the voltage controlled current source 37; and an analogue-to-digital converter (ADC) 32 configured to receive and sample a voltage signal from the instrumentation amplifier 36 and thereby generate digital voltage data. The MCU platform also includes a module 34 comprising a general purpose input/output (GPIO), a serial peripheral interface (SPI) and a clock (CLK) to interface with the DDS module 38.

4) A back-end host computer 30 (or other processing/visualisation device, such as, but not limited to, a tablet computer or smartphone) to reconstruct the data for visual display. In alternative embodiments this need not provide visualisation, and could just provide an indicator (e.g. an audible notification) instead.

A predefined stimulation waveform is stored in the MCU 31 and sent to the DAC 33 or the Direct Digital Synthesis (DDS) waveform generator 38, to cause the voltage waveform to be generated. The generated voltage waveform is then converted to current source with required slew rate and bandwidth. This differential current is directed to the target electrode pair in the probe 10 by the multiplexer 39. The other pair of electrodes in the probe 10 are set to sense the voltage induced by the impedance of the tissue. This voltage signal is amplified by the instrumentation amplifier 36 and converted into the digital domain by the ADC 32. Then the measured waveform is processed using FFT to obtain the impedance measurement at each frequency of interest. The impedance measurements of each different connection configuration (e.g. as shown in FIG. 12 or FIG. 13) are compared with each other to identify the existence of a margin within the four electrodes, and which pair of electrodes are the closest to the margin. In our example implementation, the overall processing time is under 73 ms for 4096 data points in each configuration.

Circuit Implementation

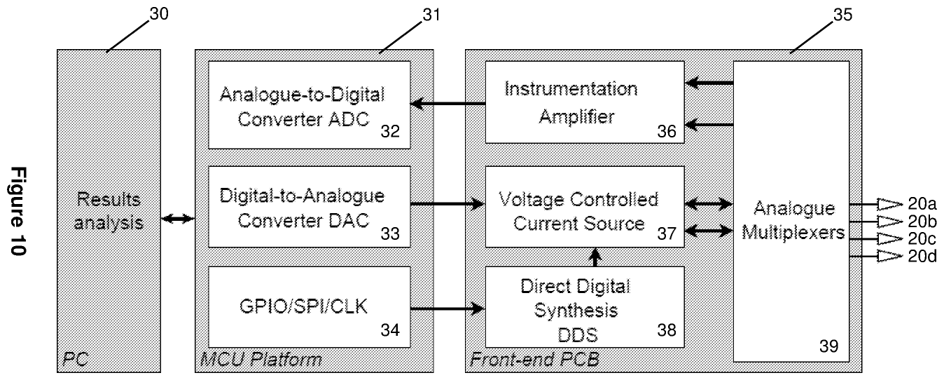

FIG. 11 shows example front-end analogue circuit schematics comprising: the voltage controlled current source 37; a voltage buffer 40; the instrumentation amplifier 36; and the multiplexer 39.

In the illustrated example, in the voltage controlled current source 37, a passive first order high pass filter (R₁, C₁) is provided to remove the DC offset of the generated waveform. A buffered differential amplifier and integrator fix the voltage across the resistor Rₐ with regard to the input voltage. This generates a current flowing from Iₒₚ to Iₒₙ accordingly. The return current is sunk or sourced by an op-amp in the voltage buffer 40 to provide a low impedance current path and allow a bias voltage to be adjusted.

To explain this further, as the voltage waveform is generated with either the DDS module 38 or the DAC 33, the voltage will have a range 0V to the maximum output voltage of the DDS 38 or DAC 33. (At this juncture, it should be noted that only one of the DDS 38 or DAC 33 may be present to generate the voltage waveform, even though both are illustrated in FIG. 10.) The high pass filter formed by R₁ and C₁ is therefore used to remove the DC offset (mean of waveform). The remaining signal is passed further into the voltage-controlled current source (VCCS) 37 to convert the voltage waveform into a current waveform.

In the illustrated example, a multi op-amp topology is used to improve the overall performance of the VCCS 37 by taking advantage of op-amps optimised in specific areas for each stage. The resulting VCCS results in a higher bandwidth current source compared to a Howland current pump, for example. Other VCCS topologies would work as well. The voltage to current ratio is set by resistor Rₐ. The amplifier on the right-hand-side of the illustrated VCCS 37 is a unity-gain amplifier; it outputs the voltage at its positive terminal with virtually zero leakage current, ensuring all of the current going through resistor R a will go into the multiplex (MUX) 39. The middle amplifier of the illustrated VCCS 37 outputs the voltage difference across resistor R^{a}. The left amplifier of the illustrated VCCS 37 completes the feedback loop and generates the required current for the voltage to current conversion.

In other words, in the illustrated example, the current source 37 is a voltage controlled current source. The processor-controlled circuitry comprises a voltage waveform generator configured to generate a voltage waveform and to supply the voltage waveform to the voltage controlled current source 37. The voltage waveform generator may comprise a digital-to-analogue converter 33 configured to receive a predefined stimulation waveform from which the voltage waveform is generated. Alternatively, the voltage waveform generator may comprise a Direct Digital Synthesis module 38, also configured to receive a predefined stimulation waveform from which the voltage waveform is generated. The voltage controlled current source 37 comprises a high pass filter configured to remove DC offset from the voltage waveform and to convert the voltage waveform to the stimulation current that is applied to the stimulation electrodes via the multiplexer 39.

The amplifier in the voltage buffer 40 offers a low impedance path for the current injected from Iₒₚ to return to the system, with the option of applying a DC voltage to further reduce DC offset on the excitation waveform (inject signal). At the same time, such an amplifier may track the bias voltage, and control the current source to compensate for the bias voltage.

The instrumentation amplifier 36 is used to measure the potential difference between the two sensing electrodes which are set for that purpose, at that moment in time, by the analogue multiplexer 39. More particularly, the instrumentation amplifier 36 amplifies the voltage difference between Vᵢₙ and Vᵢₚ. The instrumentation amplifier 36 does not have to be an off-the-shelf single package IC, and it could be formed by a combination of amplifiers to create equivalent voltage amplification or using application-specific IC.

The multiplexer 39 changeably defines each instantaneous configuration of the electrodes, and redirects Iₒₚ, Iₒₙ, Vᵢₙ, and Vᵢₚ to the designated electrodes according to the selected electrode configuration at that moment in time.

Electrode Switching Configurations

FIG. 12 shows, by way of example, four tetrapolar electrode switching configurations (A, B, C and D) sequentially constructed by the analogue multiplexer 39. In our practical implementation, the apparatus measures the impedance in each configuration in a time frame of under 22 ms, and the implemented analogue front-end circuit may have a bandwidth of up to 1 MHz. The electrodes 20a-20d are arranged in a square configuration at the distal tip of the probe 10.

In electrode switching configuration A, the stimulation current I₁ is applied from electrode 20a to electrode 20b, and the corresponding voltage V₁ is measured between electrodes 20c and 20d.

Next, in electrode switching configuration B, the stimulation current I₂ is applied from electrode 20d to electrode 20a, and the corresponding voltage V₂ is measured between electrodes 20b and 20c.

Then, in electrode switching configuration C, the stimulation current I₃ is applied from electrode 20c to electrode 20d, and the corresponding voltage V₃ is measured between electrodes 20a and 20b.

Then, in electrode switching configuration D, the stimulation current I₄ is applied from electrode 20b to electrode 20c, and the corresponding voltage V₄ is measured between electrodes 20d and 20a.

The sequence of switching configurations A-D may be repeated by the multiplexer 39 in a cyclic manner.

FIG. 13 shows how the number of different configurations with a four-electrode measurement can be extended to six whilst maintaining four electrodes, by adding further switching configurations E and F to the above-described sequence of switching configurations A-D.

In electrode switching configuration E, the stimulation current I₅ is applied from electrode 20a to electrode 20c, and the corresponding voltage V₅ is measured between electrodes 20b and 20d.

In electrode switching configuration F, the stimulation current I₆ is applied from electrode 20b to electrode 20d, and the corresponding voltage V₆ is measured between electrodes 20a and 20c.

The sequence of switching configurations A-F may be repeated by the multiplexer 39 in a cyclic manner.

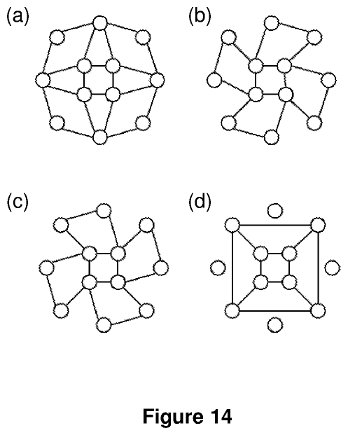

As those skilled in the art will appreciate, more than four stimulator electrodes may be used. To illustrate this, FIG. 14 provides examples ((a)-(d)) of how the aforementioned probe configuration employing four-electrode measurement can be extended to more electrodes (with each circle in the figure representing a respective electrode).

More particularly, using the understanding from the initial tetrapolar configuration probe, the number of electrodes can be increased, and a more sophisticated electrode arrangement can be formed. FIG. 14 provides examples of how the scale and complexity of the probe can be extended. With 12 electrodes, multiple tetrapolar configurations can be formed between the electrodes, greatly increasing the resolution of the measurements. Margin analysis can be performed using similar electrode placement to estimate how the margin passes through the area covered by the electrodes.

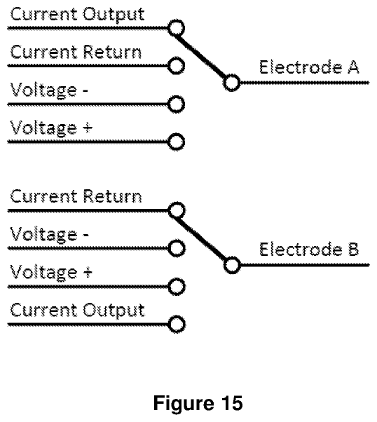

FIG. 15 schematically illustrates a multiplexer switching arrangement for supplying a current to a selected pair of stimulation electrodes (in this case, “Electrode A” and “Electrode B”) when cycling though a series of electrode configurations. Each electrode can be switched to any of the following by operation of the multiplexer 39:
“Current Output”—to supply the stimulation current to the tissue
“Current Return”—to receive the stimulation current from the tissue
“Voltage−” and “Voltage+”—the electrodes between which the corresponding voltage is measured

Waveform Characteristics

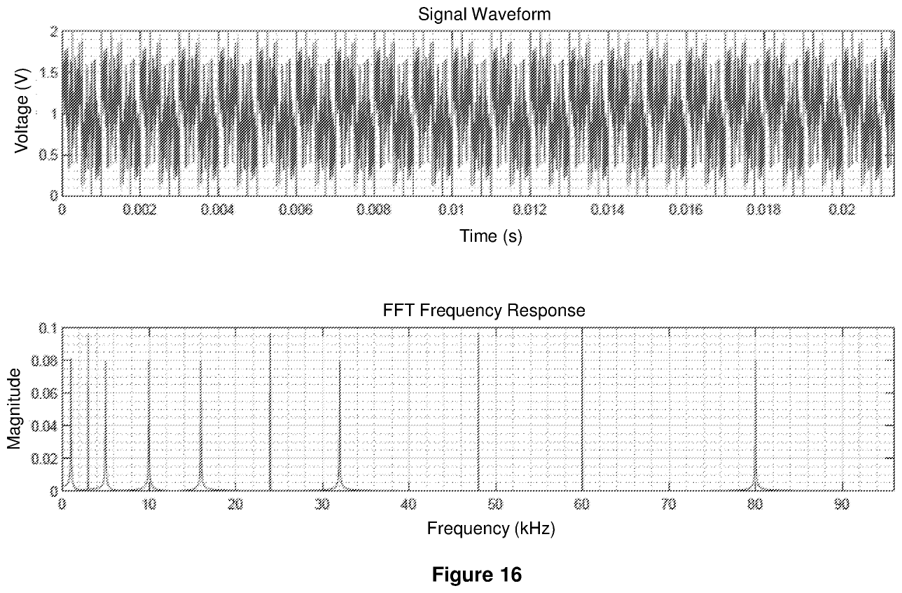

Advantageously, in the presently-preferred embodiments, the voltage waveform that is generated and supplied to the voltage controlled current source 37 (i.e. by the DAC 33 or DDS 38) comprises a mix of a plurality of different frequencies. This is illustrated in FIG. 16. The upper trace shows an example voltage waveform, whereas the lower trace shows a corresponding FFT frequency response output, showing that the voltage waveform comprises multiple frequencies.

As mentioned above, the predefined voltage waveform is converted to the stimulus current by the VCCS 37. In the illustrated example the waveform is designed to have substantially equal magnitude at the different frequencies of 1 kHz, 3 kHz, 5 kHz, 16 kHz, 24 kHz, 32 kHz, 48 kHz, 60 kHz, and 80 kHz, sampled at a frequency of 192 kHz. The number of sinusoidal waveforms and their frequencies is not fixed, but the maximum measurable sinusoidal frequency will need to be half of the sampling frequency (Nyquist sampling criterion). Advantageously a multi-frequency waveform is used such that multiple frequencies' impedance can be rapidly extracted using FFT, greatly reducing the measurement time compared to a standard chirp technique. This is because a standard chirp technique would require frequency sweeping to take place. However, the present technique employs multiple frequencies simultaneously, enabling the impedance result to be rapidly obtained, after only a small number (e.g. a few) of electrode switching cycles. The above frequencies have been chosen to keep the overall amplitude of the waveform low to maximise the dynamic range of the system and with a low slew rate to ease requirements for electronic components. The chosen frequency range may be limited due to component limitations. In addition, the phase of an individual sinusoidal waveform can be changed to further reduce the overall amplitude of the waveform.

In our example implementation, we used 4096 samples due to resource constraints by the microcontroller architecture; however, any number of samples would work. A higher number of samples would increase the frequency resolution of the calculated impedance; a lower number of samples would reduce the total measurement time. The predefined waveform may be repeated multiple times in the measurement period, such that the calculated impedance would be an averaged overall repetition of the waveform, resulting in lower noise measurement.

Operating Method

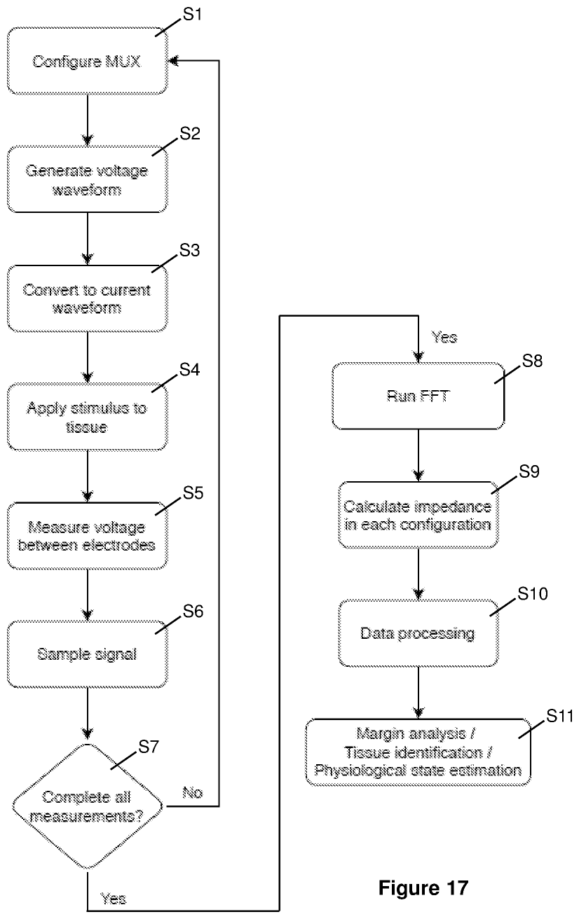

FIG. 17 provides a flow diagram illustrating the operation of the above-described bioimpedance characterisation system and measurement probe. The constituent steps are as follows:

Step S1: Configure the switching configuration of the multiplexer 39 for the designated electrode configuration for that moment in time (e.g. one of configurations A to D as shown in FIG. 12), by applying a control signal to the multiplexer 39.

Step S2: Generate the voltage waveform through the DAC 33 or DDS 38.

Step S3: Pass the voltage waveform into the high pass filter to remove DC offset, and convert to stimulation current waveform (excitation signal) in the voltage controlled current source 37.

Step S4: Apply the stimulation current waveform (excitation signal) to the tissue via the multiplexer, using one electrode for current injection, and another electrode as the current return path.

Step S5: Measure voltage between the other two electrodes using instrumentation amplifier 36 (or differential amplifier).

Step S6: Sample the measured voltage signal with the ADC 32.

Step S7: If measurements have been performed on all switching configurations, continue to step S8, otherwise, repeat the process from step S1, reconfiguring the electrode configuration to the next in the sequence (e.g. switching from configuration A to configuration B; or from B to C; or from C to D; or from D to A, until measurements have been performed at least once on each of the switching configurations).

Step S8: Convert the data type of the sampled data into floating-point (data type for running FFT) and run FFT (Fast Fourier Transform). Any suitable algorithm can be used to convert time-domain data into the frequency domain, or other means may be used to extract the instantaneous magnitude across the frequency through time (e.g. wavelet transform).

Step S9: Calculate the relative impedance of the tissue in each configuration using suitable algorithms (e.g. application of ohm's law, machine learning, networks).

Steps S10 & S11: Data processing and results analysis, performed by PC 30. Algorithms can be applied on the data generated in step S9 to perform margin analysis, tissue identification, physiological state estimation, etc., according to the surgeon's requirements.

### Experimental Demonstrations

### Example 1— Test Using Metal Plate

A saline testing setup was assembled with our example implementation of the present device, using a 3-axis cramp to hold the probe over the testing area. A 1 cm×1 cm piece of metal was placed at the centre of the glass container with 1 cm deep saline solution. A set of 6-by-6 impedance measurements were recorded using the electrodes with a 2.54 mm spacing. The electrodes used were gold-plated spring-loaded pins.

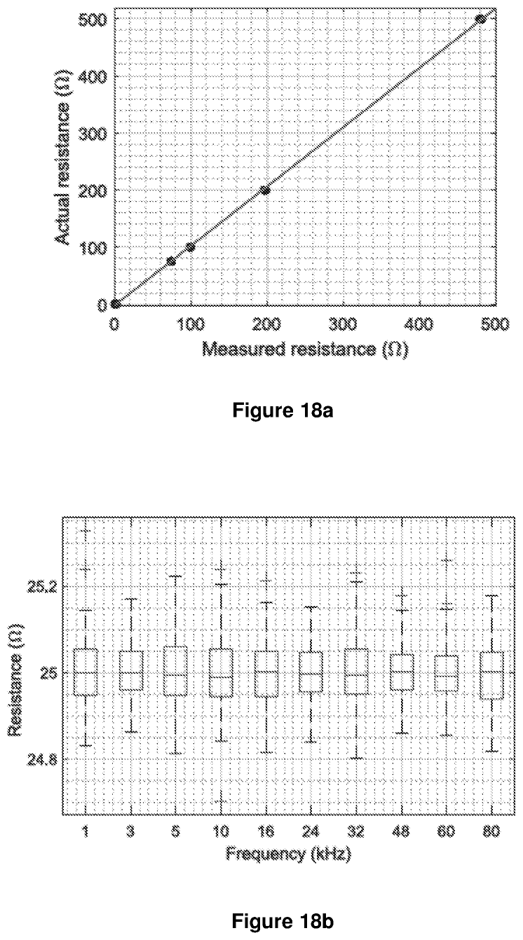

The implemented device was calibrated with a 25Ω resistor with 0.5% tolerance. The typical error of the device is below 2% with a resolution of 1Ω. FIG. 18a shows the linearity of the device, comparing measured impedance with actual impedance in respect of impedance values from 0.2Ω to 500Ω, and FIG. 18b shows the noise analysis on a 25Ω resistor. From FIG. 18a, the measurements on different resistor values have shown the device has a linearity of 0.1%. The error increases as the impedance under test decreases, as the signal approaches the noise floor.

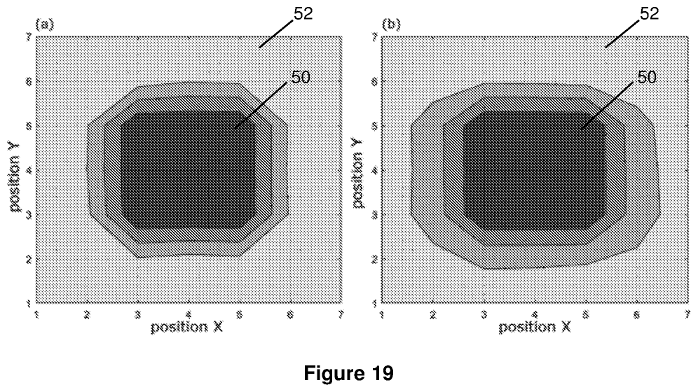

FIG. 19 shows the reconstructed resistance mapping, across and around the 1 cm×1 cm piece of metal, using (a) a commercially-available Agilent E4980A Precision LCR meter, and (b) the implemented device, respectively. The dark-shaded low impedance area denotes the metal 50 at the centre, surrounded with light-shaded (relatively high impedance) saline solution 52. Both the Agilent E4980A LCR meter and the implemented device successfully detected the location and the margin of the metal.

### Example 2

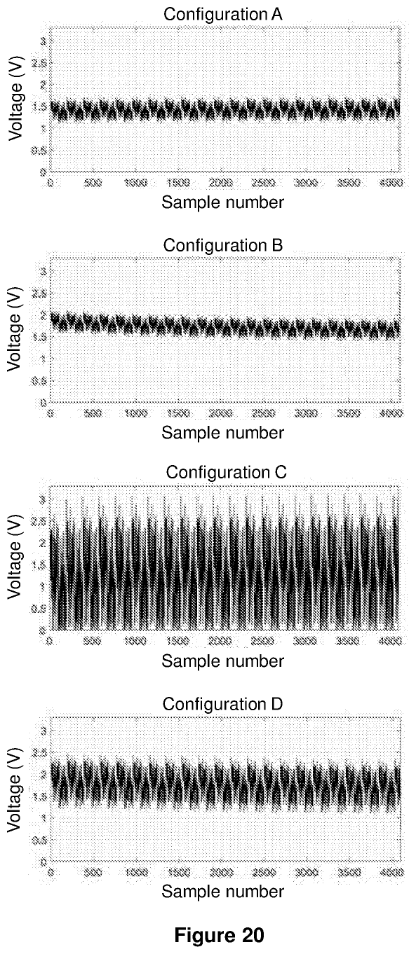

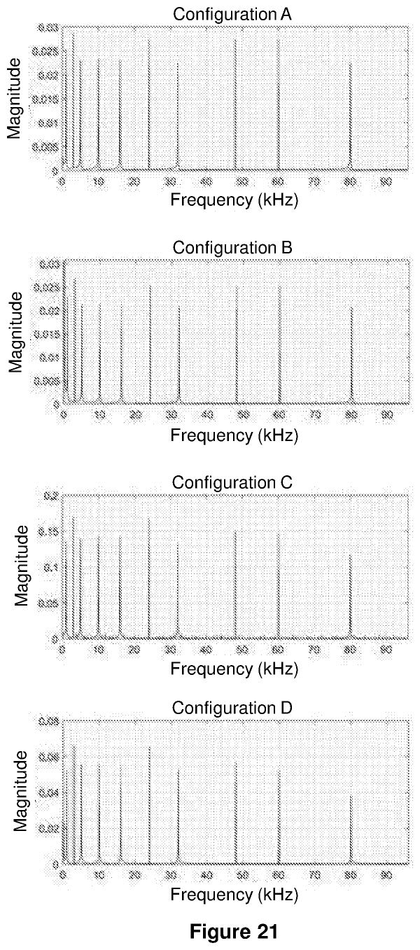

FIG. 20 illustrates four voltage waveforms (as measured by the instrumentation amplifier 36 and sampled by the ADC 32) measured from a region of biological tissue, and FIG. 21 illustrates the respective FFT frequency responses, for each of the four electrode switching configurations A-D shown in FIG. 12.

As illustrated, the bottom left electrode is denoted 20a, the bottom right electrode is 20b, the top right electrode is 20c, and the top left electrode is 20d. As in FIG. 12, in configuration C of FIG. 20 the current was injected at electrode 20c and returned via electrode 20d, and the corresponding voltage was measured across electrodes 20a and 20b. From FIG. 20 it can be seen that the measured voltage in configuration C, across electrodes 20a and 20b, is the highest compared to other configurations.

Then, in configuration D of FIG. 20, the current was injected at electrode 20b and returned via electrode 20c, and the corresponding voltage was measured across electrodes 20a and 20d. It can be seen that the measured voltage was higher than the voltages observed in configurations A and B, although not as high as in configuration C.

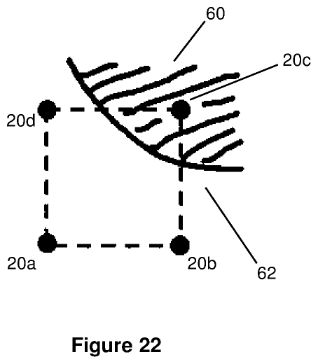

These voltage measurements can be interpreted as meaning that electrode 20c is on an area with higher bioimpedance compared to the surroundings, but the area of higher bioimpedance does not extend to any other electrodes. Such an arrangement is sketched in FIG. 22, wherein the shaded area 60 (in which electrode 20c is located) represents tissue with higher bioimpedance (e.g. different physiological state tissues), and the unshaded area 62 (in which electrodes 20a, and 20d are located) represents tissues with lower bioimpedance. The area 60 can be irregular, and the bioimpedance need not be constant across one area. This illustrates that, in this example, the tissue under electrode 20c is different from that under the other electrodes, and the differences do not reach any of the other electrodes.

### Example 3

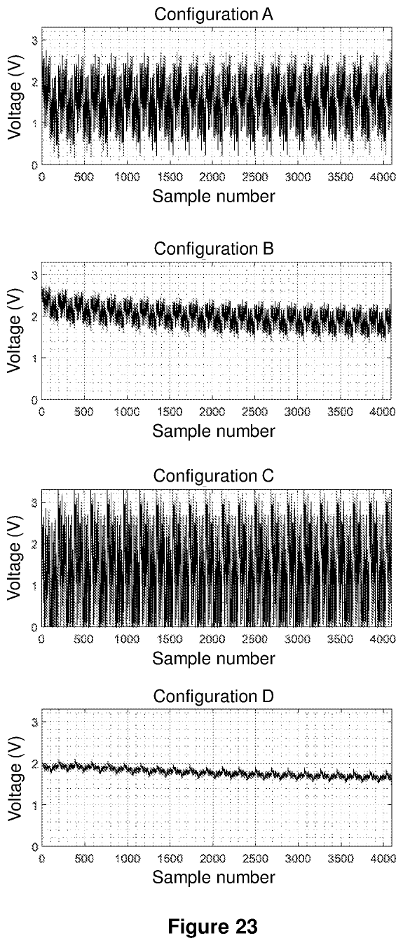

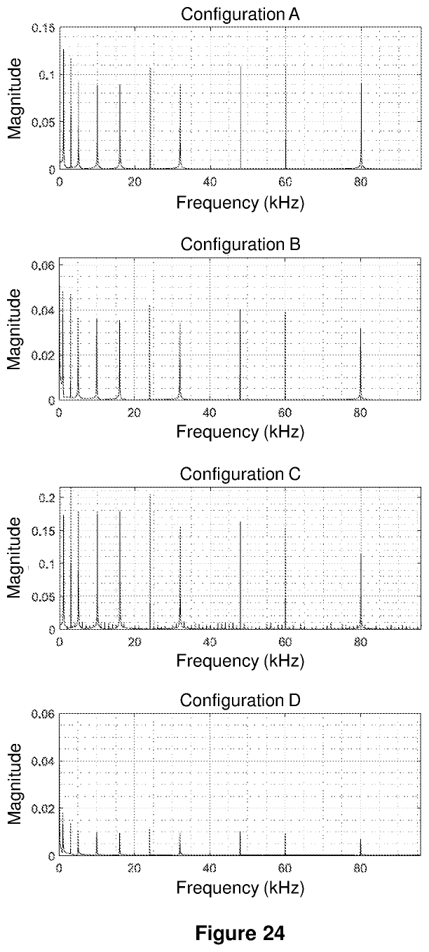

From a different region of biological tissue to that of Example 2, FIG. 23 illustrates another four measured voltage waveforms (as measured by the instrumentation amplifier 36 and sampled by the ADC 32), and FIG. 24 illustrates the respective FFT frequency responses, for each of the four electrode switching configurations A-D shown in FIG. 12.

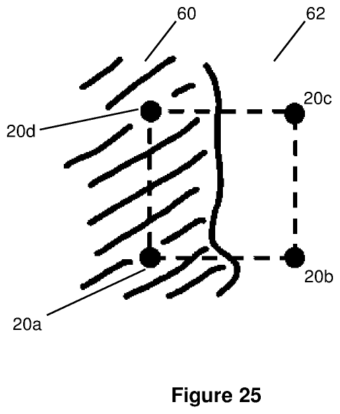

From FIG. 23 it can be seen that the measured voltage in configurations A and C are higher than those measured in configurations B and D. These voltage measurements can be interpreted as meaning that there is a tissue margin crossing the probe, such that electrodes 20a and 20d are on one side of the margin, and electrodes 20b and 20c are on the other side of the margin. Such an arrangement is sketched in FIG. 25, wherein the shaded area 60 (in which electrodes 20a and 2d are located) represents tissue with higher bioimpedance (e.g. different physiological state tissues), and the unshaded area 62 (in which electrodes 20b and 2c are located) represents tissues with lower bioimpedance.

### Example 4— Test on Rib-Eye Steak

By scanning the probe across a tissue sample and taking impedance measurements at each position using each of the four electrode configurations, the present technique may be used to calculate the bioimpedance under the probe at each position across the sample, and an impedance map can in turn be generated. (Even if just a single configuration were to be used in each position, an impedance map could still be achieved, although this would be of courser quality, and some measurements could be saturated, giving erroneous results.) Using the present four-configuration measurement technique at each position across the sample therefore provides better macro scale robustness and resolution.

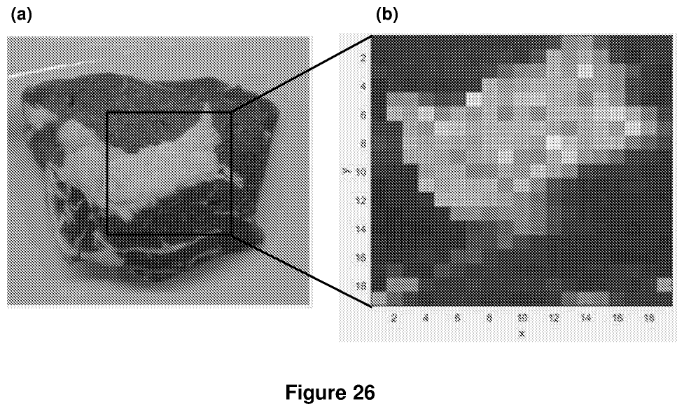

To illustrate this, the same practical implementation as used in Example 1 was used with the piece of metal replaced by a portion of rib-eye steak, as shown in image (a) of FIG. 26. Image (b) of FIG. 26 shows a reconstructed impedance map obtained using the present device. The high impedance area (having lighter shading) mainly represents the fat on the steak. The map illustrates the margin of the fat and muscle fibre within the state (the muscle fibre being darker shaded, having relatively low impedance). This demonstrates that the present apparatus is able to discriminate between different types of tissue using bioimpedance measurements.

### Conclusion of Experimental Demonstrations

In this work, a bioimpedance measuring device has been successfully implemented for performing real-time tissue analysis. By using a multi-tone sinusoid waveform, the implemented system was capable of performing impedance characterisation from 1 kHz to 80 kHz with a minimum resolution of 1Ω. The system was verified first using an electronic dummy model (metal plate), and subsequently with a biological sample (a piece of rib-eye steak under controlled temperature conditions).

The measured results demonstrated that the instrument achieved a maximum 2% error, a linearity of 0.1%, and a power consumption of 736.7 mW.

The designed instrument was compared with measurement results obtained using a commercially-available Agilent E4980A Precision LCR meter. The implemented system achieved similar performance in terms of relative impedance and reaffirmed the feasibility of the present technique. As proof of concept, it has been shown that the design is versatile to different bioimpedances. With adjustable gain and configurable waveform, the present technique can be used on various tissues and conductive solutions with various impedance ranges.

### SUMMARY

The ability to acquire real-time diagnostics of brain tissue intraoperatively represents a key goal in the field of brain tumour neurosurgery. This can greatly enhance the precision, extent and effectiveness of key surgical procedures such as those performed for brain tumour resection and biopsy. To achieve this requires a miniature, handheld tool which can perform intraoperative in situ, in-vivo characterisation of different types of tissues, e.g. normal brain tissue versus tumour tissue. In the present work we have explored the feasibility and requirements of implementing a portable impedance characterisation system for brain tumour detection. We have proposed and implemented a novel system based on PCB-based instrumentation using, for example, a square four-electrode microsurgical probe. The demonstrated system uses a digital-to-analogue converter to generate a multi-tone sinusoid waveform, and a floating bi-directional voltage-to-current converter to output the differential stimulation current to one pair of electrodes, in each of a plurality of electrode switching configurations. The other pair of electrodes in each electrode switching configuration are connected to a sensing circuit based on an instrumentation amplifier. The recorded data is pre-processed by the micro-controller and then analysed on a host computer. To evaluate the system, tetrapolar impedances were first recorded from a number of different electrode configurations to sense pre-defined resistance values. The overall system consumed 143 mA current, achieved 0.1% linearity and 15 μV noise level, with a maximum signal bandwidth of 100 kHz. Initial experimental results on tissue were subsequently carried out, including on a piece of rib-eye steak. Electrical impedance maps (EIM) and contour plots were then reconstructed to represent the impedance values in different tissue regions.

Thus, a handheld electrical ‘tumour identification’ probe has been developed which passes an electrical current through biological tissue while concurrently measuring the resistance to the flow of current produced by the tissue, i.e. the bioimpedance of the tissue. By characterising this bioimpedance in real-time, the probe enables tissue identification to differentiate normal brain tissue from abnormal brain tissue, i.e. brain tumour tissue. The probe has been embodied as a minimally-invasive neurosurgical tool, e.g. for use in microscopic and endonasal endoscope assisted neurosurgery. The latter epitomises minimal access surgery necessitating the use of light, thin, long instruments which can be easily manipulated, even with the restricted access provided by a single nasal passage.

### Modifications and Alternatives

Detailed embodiments and some possible alternatives have been described above. As those skilled in the art will appreciate, a number of modifications and further alternatives can be made to the above embodiments whilst still benefiting from the inventions embodied therein.

Notably, in the above embodiments, the current source is primarily described as being a voltage controlled current source, driven by a voltage waveform (generated by a voltage waveform generator). However, in alternative embodiments the current source may directly generate a stimulation current for driving the electrodes, with the current source being driven by a current waveform generator. The current waveform generator may for example comprise a digital-to-analogue converter configured to receive a predefined stimulation waveform from which the current waveform is generated. As in the case of the voltage waveform, such a current waveform may comprise a mix of a plurality of different frequencies, and be of substantially equal magnitude at each of the plurality of different frequencies.

Optionally the above-described probe 10 may be wireless, facilitating unencumbered manipulation of the probe by the surgeon. However, it may alternatively be connected to the control and analysis system by a cable.

Optionally the above-described probe 10 may further comprise a pressure sensor, for measuring the contact pressure between the electrodes 20a-20d and the tissue being tested. Contact pressure has been found to have an effect on the impedance measurements, and consequently measurements of the contact pressure obtained using the pressure sensor may be used to normalise the impedance measurements, thereby improving the accuracy of the impedance measurements.

Optionally the above-described probe 10 may further comprise, at the distal end of the elongate body, a blood oxygen sensor (e.g. an optical sensor to measure peripheral oxygen saturation (SpO₂)), for measuring the blood oxygen level of the tissue being tested. This may be used to notify the surgeon that a blood vessel is present within or near the tissue being tested. Accordingly, the surgeon may take suitable measures before cutting out a tumour from the tissue in question, for example.

Optionally the above-described probe 10 may be incorporate an accelerometer or inertial sensor to sense the angle of inclination (i.e. tilt) of the probe. This may be used to alert the surgeon if they inadvertently change the angle of inclination of the probe to such an extent that that it risks causing injury to the patient, for example.

### REFERENCES

[1] DeAngelis, L. M., 2001. Brain tumors. New England Journal of Medicine, 344(2), pp. 114-123.
[2] Khan, S., Mahara, A., Hyams, E. S., Schned, A. and Halter, R., 2015, March. Towards intraoperative surgical margin assessment and visualization using bioimpedance properties of the tissue. In Medical Imaging 2015: Computer-Aided Diagnosis (Vol. 9414, p. 94141C). International Society for Optics and Photonics.
[3] DePaoli D., Lemoine E., Ember K., Parent M., Prud′homme M., Cantin L., Petrecca K., Leblond F., Coto DC., 2020. Rise of Raman Spectroscopy in neurosurgery: a review. Journal of Biomedical Optics, 25(5).
[4] Morimoto, T., Kimura, S., Konishi, Y., Komaki, K., Uyama, T., Monden, Y., Kinouchi, D. Y. and Iritani, D. T., 1993. A study of the electrical bio-impedance of tumors. Journal of Investigative Surgery, 6(1), pp. 25-32.
[5] Faes, T. J. C., Van der Meij, H. A., De Munck, J. C. and Heethaar, R. M., 1999. The electric resistivity of human tissues (100 Hz-10 MHz): a meta-analysis of review studies. Physiological measurement, 20(4), p.R1.
[6] Chauveau, N., Hamzaoui, L., Rochaix, P., Rigaud, B., Voigt, J. J. and Morucci, J. P., 1999. Ex vivo discrimination between normal and pathological tissues in human breast surgical biopsies using bioimpedance spectroscopy. Annals of the New York Academy of Sciences, 873(1), pp. 42-50.
[7] Hu, S., Kang, H., Baek, Y., El Fakhri, G., Kuang, A. and Choi, H. S., 2018. Real-time imaging of brain tumor for image-guided surgery. Advanced healthcare materials, 7(16), p.1800066.
[8] Latikka, J. and Eskola, H., 2019. The resistivity of human brain tumours in vivo. Annals of Biomedical Engineering, 47(3), pp. 706-713.

1. Apparatus for discriminating between tumour tissue and non-tumorous tissue in real-time, the apparatus comprising:
a handheld bioimpedance probe having an elongate body and at least four stimulator electrodes mounted on a distal end of the elongate body, the electrodes being arranged to be held against a region of tissue in use;
a current source configured to generate a stimulation current;
a voltage sensor;
a multiplexer coupled between the current source, the voltage sensor and the stimulator electrodes and configured to changeably switch between a plurality of switching configurations, wherein, in each switching configuration, a first two of the electrodes are connected to the current source, and a second two of the electrodes are connected to the voltage sensor, the electrodes that constitute the first two electrodes and the electrodes that constitute the second two electrodes changing from one switching configuration to the next; and
processor-controlled circuitry configured to:
control the switching configuration of the multiplexer;
control the generation of the stimulation current by the current source and the application of the stimulation current to the first two electrodes of each switching configuration, such that, in use, the stimulation current flows through the tissue between the first two electrodes;
control the measurement, by the voltage sensor, of the voltage between the second two electrodes of each switching configuration, across the tissue in use;
determine, based on the applied current and the measured voltage for each switching configuration, a measure of relative impedance of the tissue for each switching configuration; and
supply such impedance measurements as output data to a data analyser to enable real-time discrimination of the tissue to be performed, based on the impedance measurements.

2. The apparatus according to claim 1, wherein the current source is a voltage controlled current source, and the processor-controlled circuitry further comprises a voltage waveform generator configured to generate a voltage waveform and to supply the voltage waveform to the current source;
optionally wherein the current source further comprises a high pass filter configured to remove DC offset from the voltage waveform and to convert the voltage waveform to the stimulation current;
optionally wherein the voltage waveform comprises a mix of a plurality of different frequencies, and optionally wherein the voltage waveform has substantially equal magnitude at each of the plurality of different frequencies;
optionally wherein the voltage controlled current source is provided on a front-end printed circuit board or integrated circuit within the probe.

3. (canceled)

4. The apparatus according to claim 1, further comprising a current waveform generator configured to generate a current waveform for directly driving the current source;
optionally wherein the current waveform comprises a mix of a plurality of different frequencies, and optionally wherein the current waveform has substantially equal magnitude at each of the plurality of different frequencies.

5. The apparatus according to claim 2, wherein the voltage waveform generator comprises a digital-to-analogue converter or a Direct Digital Synthesis module configured to receive a predefined stimulation waveform from which the voltage waveform is generated;
optionally wherein the stimulation waveform is stored in the processor-controlled circuitry;
optionally wherein the digital-to-analogue converter is provided on a microcontroller unit platform.

6. The apparatus according to claim 4, wherein the current waveform generator comprises a digital-to-analogue converter or a Direct Digital Synthesis module configured to receive a predefined stimulation waveform from which the current waveform is generated;
optionally wherein the stimulation waveform is stored in the processor-controlled circuitry;
optionally wherein the digital-to-analogue converter is provided on a microcontroller unit platform.

7. (canceled)

8. (canceled)

9. (canceled)

10. The apparatus according to claim 1, wherein the voltage sensor comprises an amplifier;
optionally wherein the voltage sensor comprises an instrumentation amplifier or a differential amplifier;
optionally wherein the amplifier is provided on a front-end printed circuit board or integrated circuit within the probe.

11. (canceled)

12. (canceled)

13. The apparatus according to claim 10, wherein the processor-controlled circuitry further comprises an analogue-to-digital converter configured to receive and sample a voltage signal from the amplifier and thereby generate digital voltage data;
optionally wherein the processor-controlled circuitry further comprises a Fast Fourier Transform processor configured to receive the digital voltage data from the analogue-to-digital converter, and to convert time-domain data into frequency-domain data and thereby extract the instantaneous magnitude of the voltage data for each of a plurality of different frequencies in the stimulation waveform;
optionally wherein the analogue-to-digital converter is provided on a microcontroller unit platform.

14. (canceled)

15. The apparatus according to claim 1, wherein the multiplexer is configured to cyclically switch between each of the plurality of switching configurations.

16. The apparatus according to claim 1, wherein the processor-controlled circuitry comprises a microcontroller;
and/or wherein the data analyser comprises a personal computer, a tablet computer or a smartphone;
and/or wherein the electrodes are spherical or hemispherical;
and/or wherein the electrodes are spring-loaded.

17. (canceled)

18. (canceled)

19. (canceled)

20. (canceled)

21. (canceled)

22. The apparatus according to claim 1, wherein the distal end of the elongate body comprises a telescopic shaft on which the electrodes are mounted.

23. The apparatus according to claim 1, wherein the probe further comprises an adjustable handle member at a proximal end of the elongate body.

24. The apparatus according to claim 23, wherein the handle member is pivotally coupled to the elongate body.

25. The apparatus according to claim 24, wherein the handle member is pivotally and retractably coupled to the elongate body by means of a linkage mechanism comprising a first joint mounted on or in the handle member, a second joint mounted on or in the elongate body, and a connecting rod that extends from the first joint to the second joint and passes through at least one of the first and second joints to an adjustable extent, wherein at least one of the first and second joints comprises a ball-and-socket joint to enable rotation in three dimensions.

26. The apparatus according to claim 25, wherein the second joint comprises a ball-and-socket joint and the rod extends through the second joint to an adjustable extent;
and/or wherein the first joint comprises a ball-and-socket joint and the rod extends through the first joint to an adjustable extent.

27. (canceled)

28. The apparatus according to claim 25 wherein, in the or each ball-and-socket joint, the surface of the respective ball part and/or the surface of the respective socket is adapted to hold the rotational position of the ball part relative to the socket in a position as set by the user.

29. The apparatus according to claim 1, wherein the probe further
comprises an operation button mounted on the elongate body, operable to activate the processor-controlled circuitry;
and/or wherein the probe further comprises depressions on the elongate body, for receiving the user's fingertips in use;
and/or wherein the probe is wireless.

30. (canceled)

31. (canceled)

32. The apparatus according to claim 1, wherein the probe further comprises a pressure sensor, for measuring the contact pressure between the electrodes and the tissue in use;
and/or wherein the probe further comprises a blood oxygen sensor at the distal end of the elongate body, for measuring the blood oxygen level of the tissue in use;
and/or wherein the probe further comprises an accelerometer or inertial sensor to sense the angle of inclination of the probe.

33. (canceled)

34. (canceled)

35. A handheld surgical tool comprising an elongate body and an adjustable handle member that is pivotally coupled to the elongate body.

36. The tool according to claim 35, wherein the handle member is pivotally and retractably coupled to the elongate body by means of a linkage mechanism comprising a first joint mounted on or in the handle member, a second joint mounted on or in the elongate body, and a connecting rod that extends from the first joint to the second joint and passes through at least one of the first and second joints to an adjustable extent, wherein at least one of the first and second joints comprises a ball-and-socket joint to enable rotation in three dimensions;
optionally wherein the second joint comprises a ball-and-socket joint and the rod extends through the second joint to an adjustable extent, and/or wherein the first joint comprises a ball-and-socket joint and the rod extends through the first joint to an adjustable extent;
optionally wherein, in the or each ball-and-socket joint, the surface of the respective ball part and/or the surface of the respective socket is adapted to hold the rotational position of the ball part relative to the socket in a position as set by the user.

37. (canceled)

38. (canceled)

39. (canceled)

40. A method of discriminating between tumour tissue and non-tumorous tissue in real-time, using the apparatus according to claim 1 to 34.
